(19) United States
(12) Patent Application Publication
(10) Pub. No.: US 2008/0226676 A1
(43) Pub. Date: Sep. 18, 2008

(54) Multivalent avian influenza vaccines and methods

(75) Inventors: Joan Plana Duran, Santa Pau (ES); Mahesh Kumar, Fort Dodge, IA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/077,044

(22) Filed: Mar. 14, 2008

Related U.S. Application Data
(60) Provisional application No. 60/918,353, filed on Mar. 16, 2007.

(51) Int. Cl. A61K 39/145

(52) U.S. Cl. 424/209.1

(57) ABSTRACT

The present invention provides vaccine compositions comprising at least two strains of avian influenza virus, wherein one of the strains has an H5 hemagglutinin subtype and the other strain has an H7 hemagglutinin subtype, and wherein at least one of the strains has an N4 neuraminidase subtype and neither strain has an N1 subtype. Also provided are vaccination methods that utilize the novel vaccine compositions of the invention. The compositions and methods of the present invention provide protection against infection with H5 and H7 influenza strains (e.g., H5N1 and H7N1) while at the same time facilitating the distinction between infected and vaccinated animals.

### BACKGROUND OF THE INVENTION

This application claims priority from copending U.S. provisional application No. 60/918,353, filed Mar. 16, 2007, the entire disclosure of which is hereby incorporated by reference.

### FIELD OF THE INVENTION

The present invention relates to the field of avian vaccines. More specifically, the invention relates to the field of multivalent avian influenza vaccines.

### BACKGROUND ART

Influenza viruses are commonly classified on the basis of the hemagglutinin (HA) and neuraminidase (NA) subtypes expressed by the viruses. (For a more thorough background on influenza viruses, see U.S. Patent Appl. Publ. No. 2006/0204976, the disclosure of which is incorporated by reference herein in its entirety). Fifteen different hemagglutinin subtypes (designated H1 through H15) and nine different neuraminidase subtypes (designated N1 to N9) have been thus far identified. An influenza virus may therefore be classified based on the hemagglutinin and neuraminidase subtype it expresses in terms such as HxNy, wherein x is a number from 1 through 15 and y is a number from 1 through 9.

HA is a viral surface glycoprotein comprising approximately 560 amino acids. It is chiefly responsible of adhesion of the viral particle to the host cell and for cell penetration in the early stages of the infection. In influenza vaccines, the HA subtype is most important in establishing immunity against viral infection. For example, a vaccine composition comprising, e.g., an inactivated or attenuated influenza virus having an H5 hemagglutinin subtype should induce immunity against infectious influenza viruses with an H5 hemagglutinin subtype.

The NA subtype, by contrast, plays a less critical role in terms of establishing immunity. Nonetheless, it has been found that the identity of the NA subtype can be useful in distinguishing vaccinated from infected animals. The ability to distinguish vaccinated animals from infected animals is important because many countries will not allow the importation of vaccinated animals that are serologically indistinguishable from infected animals. Various methodologies, known collectively as “DIVA,” allow for the distinction between infected and vaccinated animals. One such DIVA strategy relies on the identity of the NA subtype of the vaccine strain to distinguish vaccinated animals from infected animals. (See International Patent Appl. Publ. No. WO03/086453 for a description of the DIVA methodology). In particular, a vaccine virus (e.g., inactivated, attenuated, etc.) can be prepared which has an HA subtype that is the same as the HA subtype of a circulating infectious influenza virus and an NA subtype that is different from the NA subtype of the circulating infectious virus. This type of vaccine will, on the one hand, induce protective immunity against the infectious virus by virtue of its HA subtype, and on the other hand, allow workers to distinguish infected animals from vaccinated animals on the basis of the unique NA subtype. Importantly, DIVA strategies have heretofore been used and discussed primarily in the context of monovalent influenza vaccines.

Combined or multivalent (e.g., bivalent) vaccines offer a number of advantages over monovalent vaccines. One advantage of a multivalent vaccine is that fewer vaccine inoculations are required. A single preparation can be administered in one inoculation and is effective against several diseases or strains of a single disease. The decreased number of inoculations needed when vaccines are combined should lead to an increased compliance to the vaccination schedule. This in turn would likely lead to a resulting increase in vaccine coverage, which would ultimately lead to better disease control.

Bivalent avian influenza vaccines are described in U.S. Patent Appl. Publ. No. 2006/0204976 (“the '976 publication”). For example, the '976 publication provides, inter alia, an example of an H5N9+H7N1 bivalent avian influenza vaccine. This exemplary bivalent vaccine, however, does not allow one to distinguish vaccinated animals from animals that are infected with, e.g., the prevalent H5N1 virus or virulent H7N1 strain since both vaccinated and infected animals would possess the N1 neuraminidase subtype. The H5N1 strain of avian influenza is of particular concern since this strain is apparently transmissible from poultry to humans and has resulted in several human fatalities. (See, e.g., Beigel et al., N. Engl. J. Med. 353:1374-1385 (2005)). A need in the art therefore exists for bivalent avian influenza vaccines that provide protection against H5 and H7 infectious influenza viruses and also provide an easy and convenient way to distinguish infected animals (especially animals infected with H5N1 and/or H7N1) from vaccinated animals.

### BRIEF SUMMARY OF THE INVENTION

The present invention solves the aforementioned need in the art by providing a vaccine composition comprising at least two strains of avian influenza virus, wherein one of the strains has an H5 hemagglutinin subtype and the other strain has an H7 hemagglutinin subtype, and wherein at least one of the strains has an N4 neuraminidase subtype and neither strain has an N1 subtype. Also provided are vaccination methods that utilize the novel vaccine compositions of the invention.

### DETAILED DESCRIPTION OF THE INVENTION

The present invention provides vaccine compositions comprising at least a first and a second strain of avian influenza virus, wherein the first strain has an H5 hemagglutinin subtype and the second strain has an H7 hemagglutinin subtype, and wherein at least the first strain or the second strain has an N4 neuraminidase subtype, and neither the first nor the second strain has an N1 neuraminidase subtype. The absence of the N1 subtype from both strains of the vaccine composition and the presence of an N4 subtype in at least one of the strains allows workers to distinguish vaccinated animals from animals that have been infected with an infectious virus having an N1 neuraminidase subtype, e.g., H5N1 or H7N1.

As used herein, the expression “strain of influenza virus” is intended to mean one or more influenza viruses or subunit viruses having a particular hemagglutinin type (e.g., H5, H7, etc.) and a particular neuraminidase type (e.g., N2, N3, N4, N5, N6, N7, N8, N9). As used herein, a strain of influenza virus is described in terms of both its hemagglutinin and its neuraminidase subtype using the convention “HxNy,” where x is any whole number from 1 through 15, and y is any whole number from 1 through 9.

The vaccine compositions of the present invention are multivalent, e.g., bivalent, trivalent, quadravalent, etc. The term “multivalent,” as used herein, is intended to mean that the vaccine composition contains at least two strains of influenza virus that are distinguished from one another on the basis of the particular HxNy pairing that characterizes each strain. Exemplary bivalent vaccine compositions encompassed by the present invention are set forth in Table 1:

TABLE 1
EXEMPLARY BIVALENT VACCINE COMPOSITIONS

| Bivalent Vaccine | | |
|---|---|---|
| Composition No. | First Strain | Second Strain |
| 1 | H5N2 | H7N4 |
| 2 | H5N3 | H7N4 |
| 3 | H5N4 | H7N4 |
| 4 | H5N5 | H7N4 |
| 5 | H5N6 | H7N4 |
| 6 | H5N7 | H7N4 |
| 7 | H5N8 | H7N4 |
| 8 | H5N9 | H7N4 |
| 9 | H5N4 | H7N2 |
| 10 | H5N4 | H7N3 |
| 11 | H5N4 | H7N5 |
| 12 | H5N4 | H7N6 |
| 13 | H5N4 | H7N7 |
| 14 | H5N4 | H7N8 |
| 15 | H5N4 | H7N9 |

The exemplary bivalent vaccine compositions shown in Table 1 may, in certain embodiments, contain only the two strains of influenza virus listed therein and no other immunogenic components. For example, any of the bivalent compositions depicted in Table 1 (labeled 1 through 15) may contain the two listed influenza strains and no other influenza strains. In certain other embodiments, however, additional immunogenic components may be added such as, e.g., additional influenza strains and/or antigens specific for other avian diseases. For example, the vaccine compositions of the present invention may include, in addition to the two specified influenza strains, a vaccine antigen against, e.g., Marek's disease, chicken herpes virus, chicken anemia virus, Newcastle Disease virus, infectious bronchitis, infectious bursal disease virus, reovirus, etc. As explained elsewhere herein, if two or more strains of influenza virus are included in a vaccine composition of the present invention, it is preferred that at least one of the strains has an N4 neuraminidase subtype and none of the strains has an N1 neuraminidase subtype. In certain embodiments, at least one of the influenza virus strains has an H5 hemagglutinin subtype and at least one of the influenza virus strains has an H7 hemagglutinin subtype.

The influenza virus strains included in the vaccine compositions of the present invention are preferably inactivated. For purposes of the present disclosure, “inactivated viruses” encompass, inter alia, killed (e.g., noninfectious) and attenuated influenza viruses. In certain embodiments, the influenza virus strains are inactivated using available methods such as, e.g., chemical inactivation using chemical inactivating agents such as binary ethyleneimine, beta-propiolactone, formalin, gluteraldehyde, sodium dodecyl sulfate, or the like or a mixture thereof, preferably formalin. The influenza viruses of the invention may also be inactivated by, e.g., heat or psoralen in the presence of ultraviolet light. Alternatively, the viral strains used in the context of the present invention may be attenuated, e.g., live-attenuated. As used herein, the term “attenuated virus” refers to any virus that has been modified so that its pathogenicity in the intended subject is substantially reduced. Preferably, the virus is attenuated to the point that it is nonpathogenic from a clinical standpoint, e.g., animals exposed to the virus do not exhibit a statistically significant increased level of pathology relative to control animals. Exemplary live-attenuated strains of influenza viruses include, e.g., cold-adapted reassortment viruses. The viruses of the multivalent vaccine compositions of the present invention may be inactivated either prior to or subsequent to being combined with one another.

The influenza virus strains included in the vaccine compositions of the present invention may be obtained by techniques known in the art. For example, the viruses may be isolated from infected animals, such as infected chickens, and then subsequently passaged on embryonated eggs, in tissue cultures, or other suitable media to establish a master seed virus.

Recombinant influenza virus strains may be included in the vaccine compositions of the present invention. Recombinant viruses may be obtained using conventional egg-based methods in which two influenza strains with the desired features for a new vaccine (e.g., HA and/or NA subtype) are injected into an egg where their genes reassort naturally. Recombinant viruses can also be obtained by introducing multiple (e.g., two) influenza strains into tissue cultures where natural reassortment is allowed to occur. Alternatively, influenza virus strains for use in the context of the present invention may be constructed using recombinant DNA techniques which may or may not involve the use of helper viruses. Exemplary plasmid-based or “reverse genetics” techniques that can be used to make influenza virus strains for use in the context of the present invention are described, e.g., in U.S. Pat. Nos. 6,649,372, 6,887,699, 6,951,754, and in U.S. Patent Appl. Publ. Nos. 2005/0003349, 2005/0037487 and 2006/0057116.

The influenza virus strains included in the vaccine compositions of the present invention may contain one or more genetic mutations (e.g., point mutations, deletions, insertions, etc.) that attenuate or inactivate the virus. The viruses may, for example, include a partial or complete deletion of the NS1 gene. (See, e.g., U.S. Pat. Nos. 6,669,943 and 6,866,853). Mutations in any of the influenza virus gene segments are contemplated within the scope of the present invention, including one or more mutations in, e.g., the HA, NA, M1, M2, NS1, PB1, PB2, PA and/or NP genes. Preferably, any mutations included in the genome of influenza virus strains do not appreciably reduce the capacity of the vaccine viruses to generate a protective immune response against virulent challenge strains containing homologous hemagglutinin and or neuraminidase subtypes.

The vaccine compositions of the present invention may be formulated using available techniques, preferably with a pharmacologically acceptable carrier. For example, in certain embodiments, an aqueous formulation is contemplated. Such formulations utilize water, saline, or phosphate or other suitable buffers. In other embodiments, the vaccine composition is preferably a water-in-oil or oil-in-water emulsion. Also contemplated are double emulsions, often characterized as water-in-oil-in-water emulsions. The oil may help to stabilize the formulation and further function as an adjuvant or enhancer. Suitable oils include, without limitation, white oil, Drakeoil, squalane or squalene, as well as other animal, vegetable or mineral oils, whether naturally-derived or synthetic in origin.

In addition, the vaccine compositions of the present invention may contain other suitable adjuvants available in the art. These can include, e.g., aluminum hydroxide and aluminum phosphate as well as other metal salts.

Additional excipients may also be included in the vaccine compositions of the present invention, including, e.g., surfactants or other wetting agents or formulation aids. Surfactants can include the sorbitan mono-oleate esters (TWEEN® series), as well as the ethylene oxide/propylene oxide block copolymers (PLURONIC® series), as well as others available in the art. Other compounds recognized as stabilizers or preservatives may also be included in the vaccine compositions of the present invention. These compounds include, without limitation, carbohydrates such as sorbitol, mannitol, starch, sucrose, dextrin or glucose and the like, as well the preservative formalin, for example.

The vaccine compositions of the present invention may also be formulated as a dry powder, substantially free of exogenous water, which may then be reconstituted by an end user prior to administration.

The present invention also includes methods of vaccinating a bird. The methods of the invention include administering to a bird any of the multivalent influenza virus vaccine compositions described herein, including, e.g., any of the bivalent vaccine compositions set forth in Table 1. In certain embodiments of the invention, the bird is a chicken, turkey, pigeon, ostrich, game hen, squab, guinea fowl, pheasant, quail, duck, goose, or emu.

The method of administration may be selected by the skilled artisan. For instance, the vaccine composition may be administered to post-hatch, young (few days to several weeks old) birds via drinking water, spraying or eye drops. In ovo administration is also contemplated herein. For example, embryos may be inoculated, usually at about day 18-19. Other methods wherein the vaccine composition of the invention is administered parenterally, subcutaneously, peritoneally, orally, intranasally, or by other available means, preferably parenterally, more preferably intramuscularly, in effective amounts according to a schedule which may be determined according to the time of anticipated potential exposure to a carrier of the disease-causing influenza, are also within the scope of the invention.

According to the administration methods encompassed by the present invention, a dose is typically within the range of about 0.25 mL to about 2.0 mL per animal, more preferably about 0.5 mL to about 1.0 mL per animal. Thus, one, two or more doses are contemplated herein. In certain embodiments, the present invention includes vaccination methods in which a multivalent vaccine composition of the invention is administered to an animal (e.g., a bird) at a first time point and then again at a second, subsequent time point (e.g., booster vaccination). For example, the first time point may be about 1 to about 21 days of age of the animal and the second time point may be about 21 to 42 days of age of the animal.

The following examples are illustrative, but not limiting, of the method and compositions of the present invention. Other suitable modifications and adaptations of the variety of conditions and parameters normally encountered in molecular biology and chemistry which are obvious to those skilled in the art in view of the present disclosure are within the spirit and scope of the invention.

### EXAMPLES

### Example 1

### Safety and Immunogenicity of an Oil Adjuvanated Inactivated Bivalent Avian Influenza Vaccine that can be used in Diva Vaccination Strategies

### Introduction

Type A avian influenza (AI) viruses, specifically those carrying hemagglutinin H5 and/or H7, are a significant threat to poultry farms and wild birds throughout the world. Recent outbreaks of a highly pathogenic H5N1 subtype has led authorities to recommend vaccination as a means for combating the disease, especially if the vaccine allows for differentiation of infected animals from vaccinated animals (DIVA strategy).

This Example demonstrates the safety and immunogenicity of an oil adjuvanated inactivated bivalent vaccine composition containing low pathogenic H5N9 and H7N4 AI strains. One of the main advantages of this bivalent vaccine composition is that the presence of H5 and H7 hemagglutinin subtypes will confer protection against virulent strains having these hemagglutinin subtypes. In addition, the presence of the N4 neuraminidase subtype, which has a low frequency of occurrence in circulating virulent strains, will facilitate the distinction between vaccinated and infected animals using, e.g., DIVA strategies.

### Results

The bivalent vaccine composition used in this example contained strains H7N4 (A/Mallard/Italy/4810-79/04) and H5N9 (A/ck/Italy/22A/98). The strains were formulated in an oil emulsion according to standard formulation techniques. (See, e.g., U.S. Patent Appl. Publ. No. 2006/0204976).

The bivalent vaccine composition was administered to eleven white leghorn specific pathogen free (SPF) chickens at two separate time points. The first vaccination took place at the 14^{th} day of age, and the second vaccination took place at the 35^{th} day of age (21 days after the first vaccination). At the 35^{th} day of age, a first blood sample was taken from each chicken. Finally, at the 56^{th} day of age (21 days after the second vaccination) a second blood sample was taken from the chickens. This protocol is summarized in Table 2.

TABLE 2
SUMMARY OF EXPERIMENTAL PROTOCOL

| 14^{th} day of age | 35^{th} day of age | 56^{th} day of age |
|---|---|---|
| 1^{st} vaccination | 2^{nd} vaccination | 2^{nd} blood sample |
| | 1^{st} blood sample | |

Serological results from vaccinated animals were evaluated by the hemagglutination inhibition (HI) technique and are summarized in Table 3.

TABLE 3
HI ANTIBODY TITERS

| | |
|---|---|
| 21 days after | 21 days after |
| 1^{st} vaccination | 2^{nd} vaccination |

| Sera ID | H5N9 | H7N4 | H5N9 | H7N4 |
|---|---|---|---|---|
| 15 | 2 | 8 | 32 | 128 |
| 16 | 64 | 64 | 128 | 128 |
| 17 | 256 | 128 | 128 | 128 |
| 18 | 8 | 16 | 128 | 64 |
| 19 | 64 | 64 | 64 | 256 |
| 20 | 256 | 64 | 128 | 256 |
| 22 | 128 | 256 | 128 | 256 |
| 24 | 32 | 32 | 128 | 128 |
| 25 | 64 | 128 | 64 | 128 |
| 26 | 64 | 64 | 64 | 256 |
| 27 | 128 | 128 | 128 | 256 |
| geometric | 52.98 | 60.09 | 93.41 | 164.69 |
| mean | | | | |

### SUMMARY AND CONCLUSIONS

This Example demonstrates the safety and immunogenicity of an exemplary H5N9+H7N4 inactivated bivalent avian influenza vaccine formulated in an oil-containing adjuvant. Three weeks after the first vaccination, mean HI antibody titers were 52.98 for H5N9 and 60.09 for H7N4. Three weeks after a booster administration of the vaccine, mean HI antibody titers were 93.41 for H5N9 and 165 for H7N4. In addition, no remarkable adverse local or systemic reactions were observed, nor was body weight adversely affected following administration of the bivalent vaccine composition.

The inactivated bivalent H5N9+H7N4 vaccine composition used in this Example would be useful in DIVA strategy vaccination campaigns due to the presence of the N4 neuraminidase subtype which could be used to distinguish vaccinated animals from animals infected with, e.g., H5N1.

### Example 2

### Efficacy of an Inactivated Bivalent H5N9+H7N4 Vaccine Composition

In this example, the ability of a bivalent avian influenza vaccine composition to protect chickens from infection by H5N1 and/or H7N1 is evaluated.

Inactivated H5N9 and H7N4 AI strains are formulated in an oil-containing adjuvant, similar to the vaccine composition described in Example 1. Twenty SPF chickens are vaccinated intramuscularly at two weeks of age with a single dose of the bivalent vaccine formulation. A second dose of vaccine is administered to the chickens three weeks later when the chickens are five weeks of age. A group of twenty SPF chickens is maintained as unvaccinated controls.

Following the second vaccination, ten chickens from the vaccinated group and ten chickens from the unvaccinated control group are challenged with virulent H5N1 virus. The remaining ten chickens from the vaccinated group and the remaining ten chickens from the unvaccinated control group are challenged with virulent H7N1 virus. All chickens are monitored daily for serological and clinical signs of H5N1 and H7N1 infection. An effective bivalent vaccine should protect the vaccinated chickens from serological and clinical signs of H5N1 and H7N1 infection, while unvaccinated, challenged animals should exhibit significant signs of infection.

### Example 3

### Distinguishing Infected Animals from Vaccinated Animals

In this example, chickens that are vaccinated with a bivalent H5N9+H7N4 vaccine composition are distinguished from unvaccinated chickens and from chickens that are infected with a virulent H5N1 AI strain.

Inactivated H5N9 and H7N4 AI strains are formulated in an oil-containing adjuvant, similar to the vaccine composition described in Example 1. Forty SPF chickens are divided into four groups of ten chickens each. The first group is unvaccinated and unchallenged. The second group is unvaccinated and challenged with a virulent strain of H5N1. The third group is vaccinated with the bivalent H5N9+H7N4 vaccine composition and is unchallenged. The fourth group is vaccinated with the bivalent H5N9+H7N4 vaccine composition and is challenged with a virulent strain of H5N1. Similar experiments can be performed using H7N1 as a challenge strain.

Blood samples are taken from all chickens and the presence of the N1- and N4-encoding genes is assayed by nucleic acid hybridization and/or PCR-based techniques (e.g., using the methods disclosed in International Patent Appl. Publ. No. WO03/086453). Chickens in the first group (unvaccinated and unchallenged) should test negative for both neuraminidase subtypes. Chickens in the second group (unvaccinated and challenged) should test positive for the N1 neuraminidase subtype but should test negative for the N4 neuraminidase subtype. Chickens in the third group (vaccinated and unchallenged) should test positive for the N4 neuraminidase subtype but should test negative for the N1 neuraminidase subtype. Finally, chickens in the fourth group (vaccinated and challenged) should test positive for both the N1 and N4 neuraminidase subtypes. A parallel set of experiments can also be performed in which the presence of the N9-encoding gene is assayed (by e.g., nucleic acid hybridization or PCR) to identify vaccinated animals.

Thus, the bivalent vaccine composition of the present invention should provide adequate protection against virulent AI strains (e.g., H5N1 and H7N1), while at the same time allowing farmers and government officials to distinguish infected animals from vaccinated animals.

Although the foregoing invention has been described in some detail by way of illustration and example for purposes of clarity of understanding, this invention is not limited to the particular embodiments disclosed, but is intended to cover all changes and modifications that are within the spirit and scope of the invention as defined by the appended claims.

All publications and patents mentioned in this specification are indicative of the level of skill of those skilled in the art to which this invention pertains. All publications and patents are herein incorporated by reference to the same extent as if each individual publication or patent application were specifically and individually indicated to be incorporated by reference.

What is claimed is:

1. A vaccine composition comprising at least a first and a second inactivated strain of avian influenza virus,
wherein the first strain has an H5 hemagglutinin subtype and the second strain has an H7 hemagglutinin subtype; and
wherein at least the first strain or the second strain has an N4 neuraminidase subtype, and wherein neither the first nor the second strain has an N1 neuraminidase subtype.

2. The vaccine composition of claim 1, wherein the first strain has an N4 neuraminidase subtype.

3. The vaccine composition of claim 1, wherein the second strain has an N4 neuraminidase subtype.

4. The vaccine composition of claim 1, wherein both the first and the second strains have an N4 neuraminidase subtype.

5. The vaccine composition of claim 1, wherein the first strain has an N9 neuraminidase subtype and the second strain has an N4 neuraminidase subtype.

6. The vaccine composition of claim 1, wherein the first strain has an N4 neuraminidase subtype and the second strain has an N9 neuraminidase subtype.

7. The vaccine composition of claim 1, further comprising an aqueous carrier.

8. The vaccine composition of claim 7, wherein the aqueous carrier is water, saline or phosphate buffer.

9. The vaccine composition of claim 1, further comprising an oil-containing emulsion.

10. The vaccine composition of claim 9, wherein said oil-containing emulsion is an oil-in-water emulsion.

11. The vaccine composition of claim 9, wherein said oil-containing emulsion is a water-in-oil emulsion.

12. A method of vaccinating a bird, said method comprising administering to the bird a vaccine composition comprising at least a first and a second inactivated strain of avian influenza virus,
wherein the first strain has an H5 hemagglutinin subtype and the second strain has an H7 hemagglutinin subtype; and
wherein at least the first strain or the second strain has an N4 neuraminidase subtype, and wherein neither the first nor the second strain has an N1 neuraminidase subtype.

13. The method of claim 12, wherein the first strain has an N4 neuraminidase subtype.

14. The method of claim 12, wherein the second strain has an N4 neuraminidase subtype.

15. The method of claim 12, wherein both the first and the second strains have an N4 neuraminidase subtype.

16. The method of claim 12, wherein the first strain has an N9 neuraminidase subtype and the second strain has an N4 neuraminidase subtype.

17. The method of claim 12, wherein the first strain has an N4 neuraminidase subtype and the second strain has an N9 neuraminidase subtype.

18. The method of claim 12, wherein the bird is a poultry animal.

19. The method of claim 18, wherein the poultry animal is a chicken, turkey, ostrich, pigeon, game hen, squab, guinea fowl, pheasant, quail, duck, goose, or emu.

20. The method of claim 18, wherein the poultry animal is a chicken.

21. A method of vaccinating a bird, said method comprising administering a vaccine composition to the bird at a first time point followed by administering the vaccine composition to the bird at a second time point,
wherein said vaccine composition comprises a first and a second inactivated strain of avian influenza virus,
wherein the first strain has an H5 hemagglutinin subtype and the second strain has an H7 hemagglutinin subtype; and
wherein at least the first strain or the second strain has an N4 neuraminidase subtype, and wherein neither the first nor the second strain has an N1 neuraminidase subtype.

22. The method of claim 21, wherein the first time point is at 1 to 21 days of age of the bird, and wherein said second time point is at 21 to 42 days of age of the bird.

23. The method of claim 22, wherein the first time point is at 14 days of age of the bird.

24. The method of claim 22, wherein the second time point is at 35 days of age of the bird.
